(19) United States
(12) Patent Application Publication
(10) Pub. No.: US 2010/0203620 A1
(43) Pub. Date: Aug. 12, 2010

(54) APPARATUS FOR CULTURING EUCARYOTIC AND/OR PROCARYOTIC CELLS

(75) Inventors: Settimio Grimaldi, Roma (IT); Antonella Lisi, Roma (IT); Livio Giuliani, Roma (IT); Donatella Sacco, Roma (IT); Enrico D'Emilia, Velletri (IT)

Correspondence Address: Pearne & Gordon LLP, 1801 East 9th Street, Suite 1200, Cleveland, OH, 44114-3108, US

(73) Assignee: C.N.R. CONSIGLIO NAZIONALE DELLE RICERCHE, I-00185 Roma (IT)

(21) Appl. No.: 11/911,972

(22) PCT Filed: Apr. 19, 2006
(86) PCT No.: PCT/IB06/02551

§ 371 Date: Dec. 10, 2009

(30) Foreign Application Priority Data
Apr. 19, 2005 (IT) MI2005A000693

(51) Int. Cl. C12M 1/38; C12M 1/36

(52) U.S. Cl. 435/286.7; 435/286.1

(57) ABSTRACT

An apparatus for culturing eucaryotic and/or procaryotic cells comprising an incubator (10), operating means (20) to generate predetermined environmental conditions within the incubator (10), and one or more control devices (30) to control the operating means (20) depending on the desired environmental conditions; apparatus (1) further comprises an electromagnetic-insulation chamber (40) housing the incubator (10) and the operating means (20), the control devices (30) being on the contrary located externally of said insulation chamber (40).

The present invention relates to an apparatus for culturing eucaryotic and/or procaryotic cells.

More particularly, the invention concerns an apparatus for culturing eucaryotic and/or procaryotic cells under controlled electric, magnetic and electromagnetic-field conditions.

The apparatus can be also employed for studying the electric, magnetic and electromagnetic-field effects on small animals.

It is known that the apparatus of this type generally comprise an incubator within which the cells or biologic populations are grown under predetermined environmental conditions; associated with the incubator are suitable control devices adapted to create said conditions.

In more detail, a solenoid may be provided which, following a controlled electric supply, produces an electromagnetic field to which the cultures are exposed. Auxiliary devices are simultaneously employed to control temperature, carbon-dioxide concentration and/or humidity within the incubator.

A problem therefore arises concerning the electromagnetic interference between the solenoid and said auxiliary devices (that are obviously equipped with control units of the electronic or electromechanical type); in fact, the incubator, solenoid and auxiliary devices are all usually positioned at the inside of the same environment which can be magnetically insulated from the external atmosphere.

Consequently, the field generated through the solenoid and intended for culturing the cells or biologic populations present in the incubator is affected by the presence of said auxiliary devices, due to the radiation emitted by said devices during usual operation of same.

The field to which the cells or biologic populations are exposed is therefore different from the expected one and the desired growth is consequently adversely affected.

This drawback is more significant in the cases in which the “nominal” field to which the cultures within the incubator are to be exposed has a very low intensity and frequency, and therefore becomes very sensitive to any type of disturbance coming from the surrounding atmosphere.

It is an aim of the present invention to provide an apparatus for culturing eucaryotic and/or procaryotic cells enabling the electromagnetic field to which the cells are exposed within the incubator to be controlled in an accurate and reliable manner.

Another aim of the present invention is to make available an apparatus allowing a reliable reproducibility and repeatability of conditions and experimental protocols to be obtained, with particular reference to the electromagnetic fields striking on the cells or biologic populations present within the incubator.

The foregoing and further aims are substantially achieved by an apparatus for culturing eucaryotic and/or procaryotic cells according to the features recited in the appended claims.

Further features and advantages will become more apparent from the detailed description of an embodiment given by way of non-limiting example, of an apparatus for culturing eucaryotic and/or procaryotic cells. This description is taken with reference to the accompanying drawings, also given by way of non-limiting example, in which:

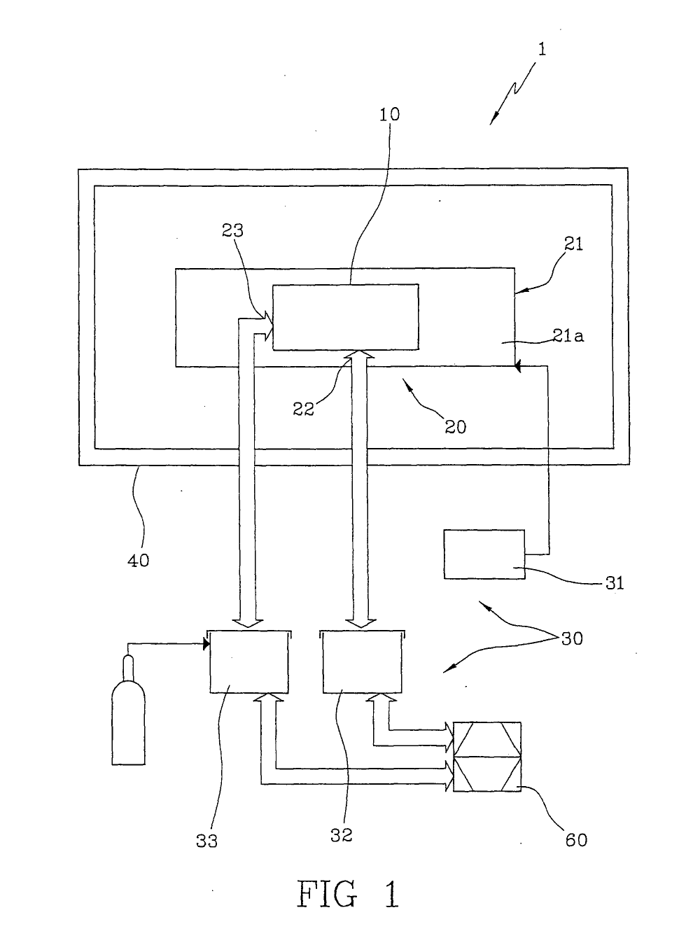

FIG. 1 shows a general block diagram of an apparatus in accordance with the invention;

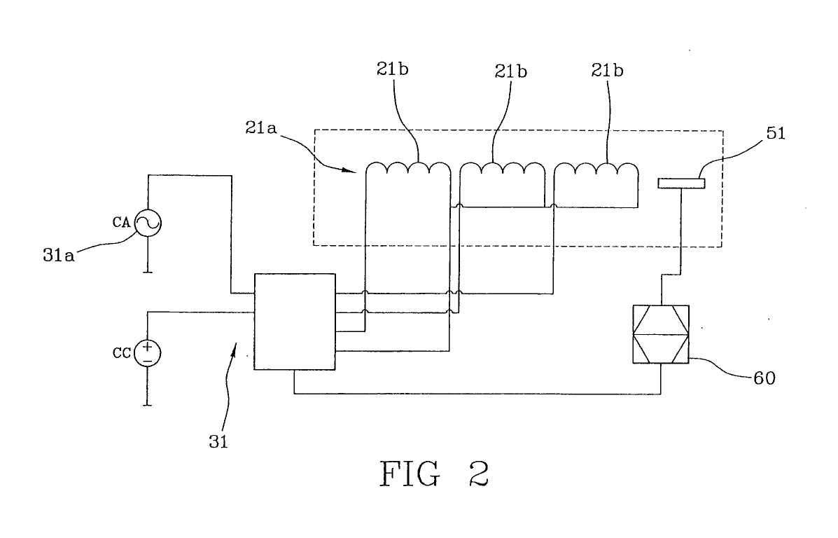

FIG. 2 shows a block diagram of a first portion of the apparatus seen in FIG. 1;

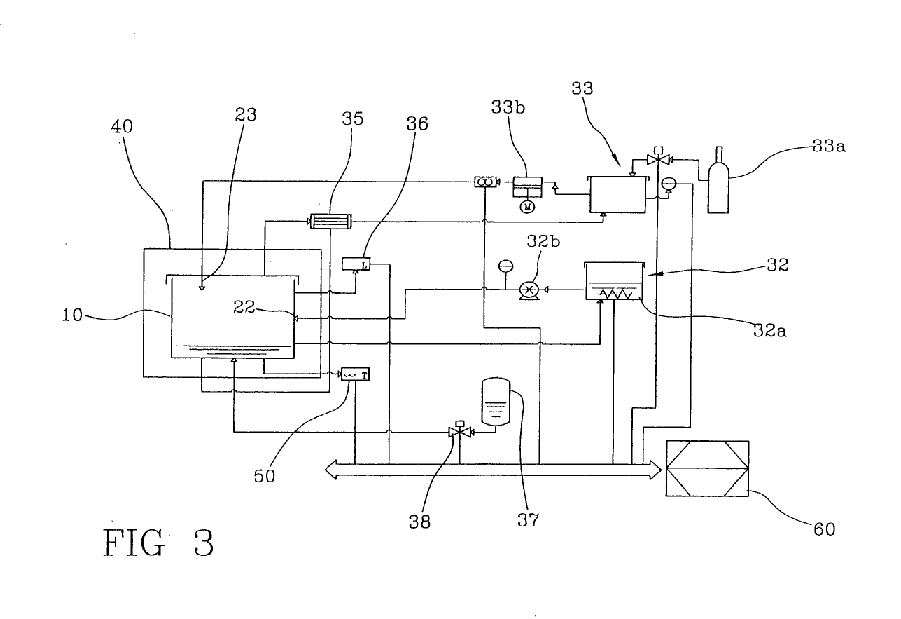

FIG. 3 shows a block diagram of a second portion of the apparatus seen in FIG. 1.

With reference to the drawings, an apparatus for culturing cells and/or growing biologic populations in accordance with the invention has been generally identified with reference numeral 1.

Apparatus 1 first of all comprises an incubator 10, preferably made of polycarbonate, that can be used for culturing both eucaryotic cells and procaryotic cells.

To this aim, within the incubator 10 pre-established environmental conditions are determined through suitable operating means 20 interlocked with respective control devices 30; in other words, the control devices 30 act on the operating means 20 to control the latter depending on the desired environmental conditions within the incubator 10.

The operating means 20 may comprise an electromagnetic-induction element 21 associated with the incubator 10 so that a predetermined electromagnetic field strikes on the cells or biologic populations present within the incubator 10 itself. In particular, the induction element 21 can be a solenoid 21a.

Preferably, the incubator is placed inside the solenoid 21a. More preferably, the incubator 10 is located at a central position in the solenoid 21a where the electromagnetic field has the maximum linearity and can be controlled with the greatest reliability.

Apparatus 1 can be equipped with a running slide (not shown in the accompanying figures) coupled with the solenoid 21a and supporting the incubator 10 to enable insertion of the latter into the solenoid 21a.

In the preferred embodiment, rigidly mounted to the slide can be an auxiliary sensor 51 to be better described in the following.

The solenoid 21a can be formed with a cylinder of plastic material around which an enameled wire defining a plurality of coils divided into several coaxial sectors is wound.

The ratio between axial (longitudinal) length and diameter of the cylinder can be at least equal to 5:1; in particular, this ratio can be at least equal to 7:1 and preferably equal to at least 10:1. In this manner, within the solenoid an electromagnetic field that is very precise as regards its geometry, i.e. in terms of spatial arrangement of the lines of force defining it, is obtained.

By way of example, the axial length of the cylinder can be of approximately 3 meters, while the diameter can be of approximately 0.3 meter.

The enameled wire can have a diameter of about 1 mm, and can form about 3000 turns around the cylinder; the coaxial (and axially adjacent) sectors into which said turns are divided can be three in number.

The induction element 21 is used, as above mentioned, to expose the cells or biologic populations present in the incubator 10 to predetermined electromagnetic fields.

For the purpose, the control devices 30 comprise a command-signal generator 31 connected to the induction element 21 (i.e. the solenoid 21a) for generation of the required electromagnetic field within the incubator 10.

Generator 31 can include several power stages (three power stages, for example) integrated into a differential amplifier configuration, so that in addition to the terrestrial magnetic field, also the electromagnetic field for exposition of the cell cultures can be reproduced.

Use of amplifiers of a differential configuration allows a reduction in the common mode disturbances.

As above mentioned, the solenoid winding 21a can be divided into a plurality of sectors 21b that are axially contiguous to and preferably coaxial with each other. In this case, these sectors 21b are provided to be controlled and operated separately from each other so that the field generated through each individual sector 21b can be added to the field generated through the other sectors in the direction defined by the longitudinal extension of the solenoid 21a.

If, on the contrary, it is a priori known that use of all available sectors (individual windings) 21b is required, these sectors can be connected to each other by means of a conductive element so that all sectors are activated simultaneously.

For production of electromagnetic fields within the solenoid 21b, use of a generator 31a of arbitrary functions is provided. Therefore static magnetic fields (simulating the terrestrial magnetic field) superposed on low-frequency electromagnetic fields can be generated, both of them being very stable.

In addition, the characteristics of the generated fields (such as intensity, frequency and waveform) can be easily measured and easily reproduced in a precise manner. Practically, fields can be obtained that have an intensity varying between few nT and 1 mT and frequencies ranging from 0.01 Hz to several kHz.

Preferably, the static magnetic field and dynamic electromagnetic field generated are parallel to each other. This choice can be useful for differentiating eucaryotic cells and in particular staminal cells.

To carry out a reliable control on the electromagnetic field present within the incubator 10, apparatus 1 can be provided with an auxiliary sensor 51 connected to a processing unit 60, which unit is in turn connected to said generator 31 to adjust the field in the incubator 10 depending on detection by the auxiliary sensor 51.

The auxiliary sensor task is to detect the electromagnetic field present in the incubator 10 and/or within the solenoid 21a; to this aim, the auxiliary sensor 51 is preferably positioned within the solenoid 21a itself.

Practically, the auxiliary sensor 51 can be an isotropic-field sensor and preferably has a resolution in the nT order.

The processing unit 60 is practically the system computer with which the different devices of apparatus 1 are interlocked.

The operating means 20 may further comprise a heat-exchange unit 22 to define the temperature within the incubator 10. The heat-exchange unit 22 can be a pipe coil formed with a rubber tube through which forced hot water runs for achieving the pre-set temperature in a controlled atmosphere.

The control devices 30 also comprise a thermal-control block 32 operatively active on said heat-exchange unit 22 to regulate the thermal energy transferred to said unit 22.

Practically the block 32 can comprise a vat 32a inside which the water for achieving said pre-set temperature is heated, and a positive-displacement pump 32b combined with a by-pass valve by which circulation through the plant of the suitably heated water is caused at a constant pressure.

In addition to the above, apparatus 1 can be provided with a main sensor 50 to detect the temperature and/or humidity within the incubator 10.

For temperature detection, the main sensor 50 can comprise a thermosensitive element such as a PTC having a resolution of about 0.05° C.

For humidity detection, the main sensor 50 can comprise a capacitive element provided with a dielectric varying its insulation features depending on the humidity present; accuracy of this capacitive element is preferably of 2-3%.

Based on said detection, the processing unit 60 drives operation of said thermal-control block 32 to regulate the thermal energy transferred to the heat-exchange unit 22 and thus obtain the desired temperature within the incubator 10.

The operating means 20 can further comprise a delivery element 23 to deliver a mixture of predetermined gases to the incubator 10; correspondingly, the control devices 30 comprise a controlled mixer 33 connected to the delivery element 23 to define the amount and/or concentration of said mixture. The mixture delivered to the incubator 10 preferably comprises carbon dioxide, possibly admixed with air.

In this manner it is possible to obtain a controlled atmosphere in the incubator 10, which atmosphere also has a predetermined amount of carbon dioxide (5%, for example); this gas is drawn from a bottle 33a and mixed in the right proportions by said controlled mixer 33.

Then the mixture is sent to the incubator 10, by a recirculation system, through a dual diaphragm pump 33b; a by-pass valve carries out regulation of the flow rate until a predetermined value (20 litres/minute, for example).

The mixture is injected into the incubator 10 through a diffuser (defining said delivery element 23) and drawn therefrom through a plurality of orifices (5 orifices, for example). Then the mixture is sent to a condenser that will separate water from the gaseous mixture and send water back to the incubator 10.

At the bottom of incubator 10 a certain amount of water may be also present for producing the relative humidity necessary to obtain the desired controlled atmosphere (approximately 90%).

A level sensor 36 sends the processing unit 60 a signal representing the water amount currently present in incubator 10; through a tank 37 and a solenoid valve 38 this level is adjusted depending on the requirements of each specific case.

Apparatus 1 further comprises an electromagnetic-insulation chamber 40 housing the incubator 10 and the operating means 20; in other words, the incubator 10, solenoid 21a, heat-exchange unit 22 and delivery element 23 are located internally of chamber 40.

Conversely, the control devices 30 (i.e. the command-signal generator 31, thermal-control block 32 and controlled mixer 33) are positioned externally of the insulation chamber 40.

The insulation chamber 40 is preferably made of an nonmagnetic material and its task is to ensure full electric and magnetic insulation of that which is contained therein relative to any outer source of magnetic or electromagnetic nature.

In this manner, the radiation emitted from the control devices 30 during operation thereof cannot affect the electromagnetic field generated within the incubator 10, thereby enabling precise and easily repeatable static magnetic and electromagnetic fields to be obtained.

The invention achieves important advantages.

First of all, due to the insulation obtained within chamber 40, electromagnetic fields also of very reduced intensities and frequencies can be obtained in the incubator without the features and main parameters of these fields being impaired by disturbances or noise.

In addition, exactly due to the accuracy achieved in generating the static magnetic and/or electromagnetic fields within the incubator, a great reliability is reached as regards repeatability of the experimental protocols used to define the environmental conditions to be adopted for the cells or biologic populations' growth.

Another advantage resides in that, by virtue of the above described construction choices, a very precise magnetic field from a geometric point of view (i.e. relative to the direction of the field itself) can be obtained within the solenoid, and therefore within the incubator.

1-13. (canceled)

14. An apparatus for culturing eucaryotic and/or procaryotic cells, comprising:
an incubator (10) for culturing the cells;
operating means (20) to generate predetermined environmental conditions within said incubator (10);
one or more control devices (30) acting on said operating means (20) to control the latter depending on said environmental conditions;
an electromagnetic-insulation chamber (40) housing at least said incubator (10) and said operating means (20),
wherein said one or more control devices (30) are located externally of said insulation chamber (40).

15. An apparatus as claimed in claim 14, wherein said operating means (20) comprises an electromagnetic-induction element (21) associated with said incubator (10) so that a predetermined magnetic field strikes on said cells.

16. An apparatus as claimed in claim 15, wherein said induction element (21) comprises a solenoid (21a), said incubator (10) preferably being positioned within said solenoid (21a).

17. An apparatus as claimed in claim 15, wherein said one or more control devices (30) comprise a command-signal generator (31) connected to said induction element (21) to produce said predetermined electromagnetic field.

18. An apparatus as claimed in claim 14, wherein said operating means (20) comprises a heat-exchange unit (22) designed to obtain a desired temperature within said incubator (10).

19. An apparatus as claimed in claim 18, wherein said one or more control devices (30) comprise a thermal-control block (32) operatively acting on said heat-exchange unit (22) to regulate the thermal energy transferred to said heat-exchange unit (22).

20. An apparatus as claimed in claim 14, wherein said operating means (20) comprises a delivery element (23) to deliver a mixture of predetermined gases to said incubator (10).

21. An apparatus as claimed in claim 20, wherein said predetermined gases comprise at least carbon dioxide.

22. An apparatus as claimed in claim 20, wherein said one or more control devices (30) comprise a controlled mixer (33) connected to said delivery element (23) to define the amount and/or concentration of said mixture to be delivered to said incubator (10).

23. An apparatus as claimed in claim 14, further comprising a main sensor (50) to detect the temperature and/or humidity within said incubator (10), said thermal-control block (32) adjusting said thermal energy depending on detection by said main sensor (50).

24. An apparatus as claimed in claim 23, further comprising a processing unit (60) at least connected to said thermal-control block (32) and controlled mixer (33) to manage operation of same, said processing unit (60) being preferably positioned externally of said insulation chamber (40).

25. An apparatus as claimed in claim 14, further comprising an auxiliary sensor (51) to detect the electromagnetic field in said incubator (10).

26. An apparatus as claimed in claim 25, wherein said command-signal generator (31) regulates production of said electromagnetic field depending on the detection carried out by said auxiliary sensor (51).
